(19) United States
(12) Patent Application Publication
(10) Pub. No.: US 2010/0310545 A1
(43) Pub. Date: Dec. 9, 2010

(54) Dry treatment mixture and methods of production and use

(76) Inventor: Edward Kolos, Lake Worth, FL (US)

Correspondence Address: OLTMAN, FLYNN & KUBLER, 915 MIDDLE RIVER DRIVE, SUITE 415, FORT LAUDERDALE, FL, 33304, US

(21) Appl. No.: 12/455,695

(22) Filed: Jun. 5, 2009

(51) Int. Cl. A61K 38/47; A61P 31/02

(52) U.S. Cl. 424/94.61

(57) ABSTRACT

A dry treatment mixture is provided including: a quantity of a dry formulation of a salt; and a quantity of a dry formulation of lysozyme. The salt preferably is sea salt. The dry treatment mixture preferably additionally includes a dry formulation of lactoferrin. A method of producing a dry treatment mixture preferably includes the steps of: providing a quantity of a dry formulation of salt in one of powder form and granular form; providing a quantity of a dry formulation of lysozyme; and mixing the quantity of a dry formulation of salt and the quantity of a dry formulation of lysozyme together to form a dry treatment mixture. The method optionally includes the yet additional step of compressing a quantity of the mixture in a mold to form a mixture pill or of encapsulating the quantity of the mixture in a capsule for oral administration, or impregnating a bandage with the mixture for topical administration.

### BACKGROUND OF THE INVENTION

1. Field of the Invention

The present invention relates generally to the field of treatments for wounds and general good health of cells. More specifically the present invention relates to a salt and enzyme dry treatment mixture for oral and topical administration to a person or to an animal for cleaning, healing, and to methods of producing the mixture and of using the mixture for treatment such as in or on an affected area of the skin of a human or of an animal whether in the form of a mammal, reptile or bird.

The mixture specifically includes a dry formulation of salt, which is preferably sea salt, and includes a dry formulation of the enzyme known as lysozyme, and optionally additionally includes a dry formulation of lactoferrin, and any of a variety of additional chemical or natural components such as essential elements, minerals, antibacterial, antibiotic, antifungal, antiviral and anti-inflammatory substances, mixed in desired percentages by weight. The dry treatment mixture preferably is provided in one of powder form, pill form, capsule form or in an impregnated bandage.

The method of producing the mixture preferably includes the steps of: providing a quantity of dry formulation salt in powder form, which may be granular; providing a quantity of one of: a dry lysozyme in powder form, the quantity being selected to be in a particular desired proportion to the selected quantity of dry formulation salt; and mixing the quantity of salt and the quantity of lysozyme together to form a mixture. The selected quantities of the dry formulation of salt and of the dry formulation of lysozyme preferably are defined in parts. The mixture proportions preferably are such that there is a substantially larger part of salt than of lysozyme. The powder forms of the quantity of salt and the quantity of enzyme preferably are micro-sized, and optionally is reduced to still smaller particles consistent with nano-technology for ready absorption into the blood or dissolved in a solution. The salt preferably is a dry formulation of sea salt, and the sea salt preferably is removed from sea water through the process of distillation. A dry formulation of the enzyme lactoferrin optionally is added. The method preferably includes the additional step of compressing the mixture in a mold to form a mixture pill for oral administration either by swallowing the pill or by dissolving the pill in water and drinking the water or using it topically. The method alternatively includes the additional step of encapsulating the mixture in a capsule for oral administration. The method still alternatively includes the additional step of impregnating a bandage with the mixture for topical administration. The method alternatively includes the step of permitting the mixture to remain in its powder form for topical administration.

2. Description of the Prior Art

There have long been cleansing solutions such as alcohol, hydrogen peroxide and antiseptics such as methylate for decontaminating and otherwise treating skin abrasions and lacerations, such as those produced during body piercing, and for application to infected or inflamed inner throat membranes. Problems with these prior antiseptics have been that they often contain harsh and complex chemicals unfamiliar to consumers which can cause pain upon contact with wounds.

It is thus an object of the present invention to provide a dry treatment mixture having cleaning and healing properties which is safe while reducing bumps and scar tissue, reducing itching and increasing osmosis or circulation to the treated area.

It is another object of the present invention to provide such a dry treatment mixture which is suitable for topical administration such as application to pierced skin areas including those produced by body piercing or stitches closing a wound, to mouth sores and pressure ulcers, to acne and to infected membranes of the throat, abscessed teeth, infected gums and canker sores, for tonsillitis, burn, diabetic ulcer, skin fungus, skin infection, skin wound and insect bites, and for oral administration in pill or capsule form or in powder form dissolved in a liquid form or impregnated in a bandage for wounds, jelly fish stings, insect bites and acne.

It is still another object of the present invention to provide such a dry treatment mixture which comprises components with which consumers are familiar and comfortable, and which are safe, natural, common, mild, pure and sterile.

It is yet another object of the present invention to provide such a dry treatment mixture which causes little or no stinging when applied topically to a wound.

It is finally an object of the present invention to provide such a dry treatment mixture which can be administered both to humans and to animals.

### SUMMARY OF THE INVENTION

The present invention accomplishes the above-stated objectives, as well as others, as may be determined by a fair reading and interpretation of the entire specification.

A dry treatment mixture comprising: a quantity of a dry formulation of a salt; and a quantity of a dry formulation of lysozyme. The dry treatment mixture preferably additionally includes a dry formulation of lactoferrin. The dry treatment mixture preferably still additionally includes a dry formulation of at least one of: an antibacterial enzyme, a co-enzyme, an herb, a natural acid, an antiseptic, an organic solvent, a natural oil, methylsulfonylmethane, dimethyl sulfoxide, an antibiotic and fluoride.

A dry treatment mixture is further provided, including: a quantity of a dry formulation of sea salt; and a quantity of a dry formulation of lysozyme.

A method of producing a dry treatment mixture preferably includes the steps of: providing a quantity of a dry formulation of salt in one of powder form and granular form; providing a quantity of a dry formulation of lysozyme; and mixing the quantity of a dry formulation of salt and the quantity of a dry formulation of lysozyme together to form a dry treatment mixture.

The quantity of a dry formulation of salt and the quantity of a dry formulation of lysozyme optionally are both in the form of one of: micro-sized powder and nano-sized powder. The quantity of a dry formulation salt preferably is a dry formulation of sea salt.

The method preferably includes the additional step of distilling or dehydrating the quantity of a dry formulation of sea salt extracted from sea water. The method preferably includes the still additional step of adding a quantity of a dry formulation of lactoferrin to the mixture. The mixture preferably includes the still further step of adding a quantity of a dry formulation of at least one of: an antibacterial enzyme, a co-enzyme, an herb, a natural acid, an artificial acid, an antiseptic, an organic solvent, a natural oil, an extract, methylsulfonylmethane, dimethyl sulfoxide, an antibiotic and fluoride to the mixture. The method optionally includes the yet additional step of compressing a quantity of the mixture in a mold to form a mixture pill for oral administration. The method optionally additionally includes the step of dissolving the mixture pill in a quantity of water. The method optionally includes additional steps of: providing a capsule; and encapsulating a quantity of the mixture in the capsule for oral administration. The method optionally includes the additional step of impregnating a bandage with the mixture for topical administration. The method yet optionally includes the step of permitting the mixture to remain in its powder form for topical administration.

### BRIEF DESCRIPTION OF THE DRAWINGS

Various other objects, advantages, and features of the invention will become apparent to those skilled in the art from the following discussion taken in conjunction with the following drawings, in which:

FIG. 1 is a perspective view of a mortar and pestle which may be used to mix and grind the present dry treatment mixture, and of a quantity of the resulting dry mixture piled beside the mortar and pestle.

FIG. 2 illustrates the mixture being formed into a mixture pill.

FIG. 3 illustrates is a capsule being filled with the mixture.

FIG. 4 is a perspective view of a bandage impregnated with the mixture.

FIG. 5 is a block diagram of the process of making the present dry treatment mixture.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference is now made to the drawings, wherein like characteristics and features of the present invention shown in the various FIGURES are designated by the same reference numerals.

### First Preferred Embodiment

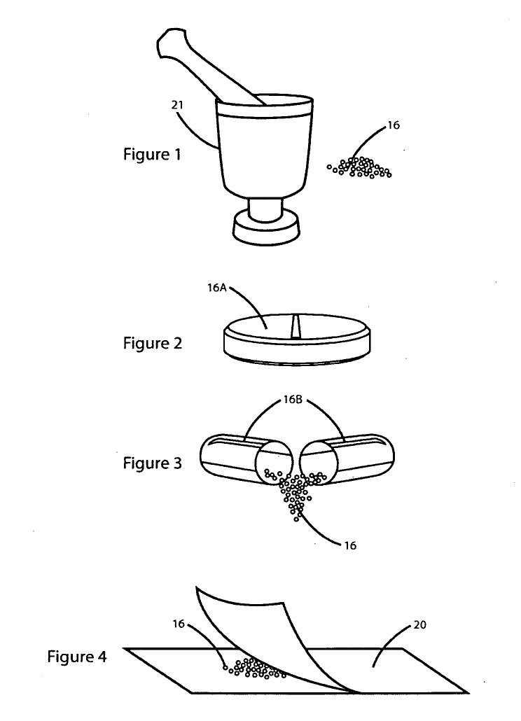

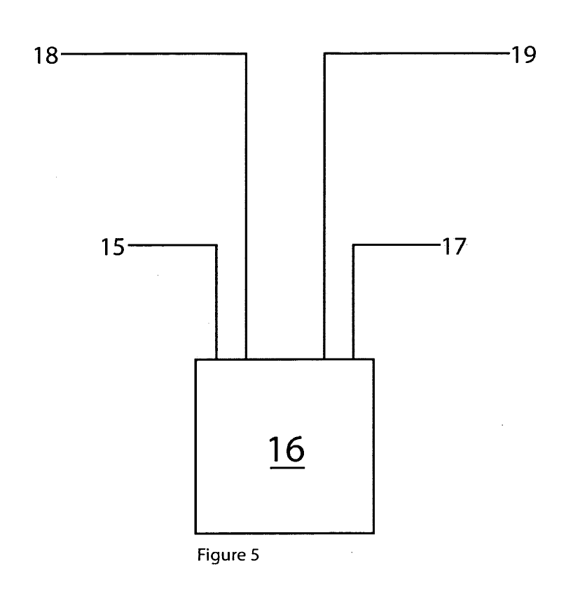

Referring to FIGS. 1-5, a dry treatment mixture 16 for topical as well as oral administration to a human body or to an animal body for cleaning and healing, such as an area subjected to a diabetic ulcer, skin fungus, bacterial infection, skin wound, rash or virus is disclosed.

The mixture 16 specifically includes dry salt 15, which is preferably a dry formulation of sea salt 15, and includes a dry formulation of a particular enzyme known as lysozyme 17, and optionally additionally includes a dry formulation of lactoferrin 19, and any of a variety of additional chemical or natural components such as essential elements, minerals, antibacterial, antibiotic, antifungal, antiviral and anti-inflammatory substances, mixed in desired percentages or ranges of percentages by weight. Sea salt 15 is understood to include 82 trace elements and sea minerals. An enzyme is defined as a peptide chain combined with an amino acid, the enzyme acting to kill harmful bacteria and thus functioning as an antibiotic. The dry treatment mixture 16 preferably is provided in one of powder form, pill 16A form, capsule 16B form or impregnated bandage 20 form.

### Method

In practicing the invention, the following method may be used. The method of producing the mixture 16 preferably includes the steps of: providing a quantity of dry formulation salt 15 in powder form, which may be granular; providing a quantity of one of: a dry lysozyme 17 in powder form, the quantity being selected to be in a particular desired proportion to the selected quantity of dry formulation salt 15; and mixing the quantity of salt 15 and the quantity of lysozyme 17 together to form a mixture 16. The selected quantities of the dry formulation of salt 15 and of the dry formulation of lysozyme 17 preferably are defined in parts. The mixture 16 proportions preferably are such that there is a substantially larger part of salt 15 than of lysozyme 17. The powder forms of the quantity of salt 15 and the quantity of enzyme preferably are micro-sized, and optionally is reduced to still smaller particles consistent with nano-technology for ready absorption into the blood. The salt 15 preferably is a dry formulation of sea salt 15, and the sea salt 15 preferably is removed from sea water through the process of distillation. A dry formulation of the enzyme lactoferrin optionally is added. The method preferably includes the additional step of compressing the mixture 16 in a mold to form a mixture pill 16A for oral administration either by swallowing the pill 16A or by dissolving the pill 16A in water and drinking the water. The method alternatively includes the additional step of encapsulating the mixture 16 in a capsule 16B for oral administration. The method still alternatively includes the additional step of impregnating a bandage 20 with the mixture 16 for topical administration. The method alternatively includes the step of permitting the mixture 16 to remain in its powder form for topical administration.

### The Components

The salt 15 component of the present mixture 16 may be sodium or salt, and preferably sea salt. Where salt 15 is sea salt, it preferably is a commercially available product used by many chefs as a condiment or seasoning to replace common table salt, and it has the purity required for human ingestion. The minerals and elements of sea salts are richer and more abundant than those of common table salt. Where the dry treatment mixture 16 is mixed with water, the water may be reverse osmosis water, purified water, tap or distilled water, or may be ocean water.

The components of the present treatment mixture 16, such as sea salt 15, and lysozyme 17 are of very high purity to guard against infection or other harm to a wound or the surrounding skin area during the healing process at the affected area.

The lysozyme 17 component is an enzyme which is harmless to and naturally produced by the human body. Lysozyme 17 is found in tears, sweat, saliva and urine and is capable of fighting infection by killing bacteria for periods of four to six hours. This enzyme also forms a shield around healthy cells to protect them from bacteria.

Other preferably components include: collagen 18 or by-product of an animal, antibacterial enzymes, antibiotics, lactoferrin, herbs, natural and artificial acids and antiseptics, organic solvents, all natural oils methylsulfonylmethane (MSM), dimethyl sulfoxide (DSMO), an antibiotic and fluoride 19. Examples of contemplated antibacterial enzymes include lactofferin, lactenin, and lactoperoxidase. Examples of contemplated natural oils include oregano oil, lavender, tea tree, lemon grass, clove oil and orange oil. Examples of natural and artificial acids include mandelis acid and ascorbic acid. Contemplated examples of antiseptics include bacitracin zinc, vitamins C, B and spearmint. Examples of contemplated organic solvents include benzene, carbon tetrachloride, trichloroethylene, 2-ethoxyethanol, 2-methoxyethanol, methyl chloride, n-hexane, tetrachloroethylene, toluene, xylene, perchloroethylene and glycol ethers. Examples of herbs contemplated as components of the present mixture include witchhazel (hamamelis virginiana linn), candrographis, astragalus, bistort, barberry, aconite, bryonia, chamomile, eucalyptus, echinacia, elderberry, elenthero, sage, sandalwood, schisandra, St. John's wort, lavender, licorice, lemon balm, liguitruor, lomatium, ginseng, hyssop, hepar sulph, garlic, goldenseal, green tea, grape, maitake, myrrh, olive leaf, onion, pand arco, Patchouli, picrorhiza, reishi, rosemary, spongia, tea tree, tyme, usnea group and wild indigo. Contemplated groups include usnea, ashwagandha and ligustruin.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

I claim as my invention:

1. A dry treatment mixture comprising:
a quantity of a dry formulation of a salt;
and a quantity of a dry formulation of lysozyme.

2. The dry treatment mixture of claim 1, additionally comprising a dry formulation of lactoferrin.

3. The dry treatment mixture of claim 1, additionally comprising a dry formulation of at least one of: an antibacterial enzyme, a co-enzyme, an herb, a natural acid and an artificial acid, an antiseptic, an organic solvent, a natural oil, methylsulfonylmethane, dimethyl sulfoxide, an antibiotic and fluoride.

4. The dry treatment mixture of claim 1, wherein said salt is sodium.

5. The dry treatment mixture of claim 1, additionally comprising a quantity of collagen.

6. A dry treatment mixture comprising:
a quantity of a dry formulation of sea salt;
and a quantity of a dry formulation of lysozyme.

7. A method of producing a dry treatment mixture, comprising the steps of:
providing a quantity of a dry formulation of salt in one of powder form and granular form;
providing a quantity of a dry formulation of lysozyme;
and mixing the quantity of a dry formulation of salt and the quantity of a dry formulation of lysozyme together to form a dry treatment mixture.

8. The method of claim 7, wherein the quantity of a dry formulation of salt and the quantity of a dry formulation of lysozyme are both in the form of one of: micro-sized powder and nano-sized powder.

9. The method of claim 7, wherein the quantity of a dry formulation salt is a dry formulation of sea salt.

10. The method of claim 7, wherein the quantity of a dry formulation of lysozyme is a dry formulation of lactoferrin.

11. The method of claim 7, comprising the additional step of adding a quantity of a dry formulation of lactoferrin to the mixture.

12. The method of claim 7, comprising the additional step of adding a quantity of a dry formulation of at least one of: an antibacterial enzyme, a co-enzyme, an herb, a natural acid, an artificial acid, an antiseptic, an organic solvent, a natural oil, methylsulfonylmethane, dimethyl sulfoxide, an antibiotic and fluoride to the mixture.

13. The method of claim 7, comprising the additional step of compressing a quantity of the mixture in a mold to form a mixture pill for oral administration.

14. The method of claim 13, comprising the additional step of dissolving the mixture pill in a quantity of water.

15. The method of claim 7, comprising the additional steps of:
providing a capsule;
and encapsulating a quantity of the mixture in the capsule for oral administration.

16. The method of claim 7, comprising the additional step of impregnating a bandage with the mixture for topical administration.

17. The method of claim 7, comprising the step of permitting the mixture to remain in its powder form for topical administration.

18. The method of claim 7, comprising the step of adding a quantity of a dry formulation of collagen.
